(19) United States
(12) Patent Application Publication
(10) Pub. No.: US 2020/0098471 A1
(43) Pub. Date: Mar. 26, 2020

(54) ACTIONS BASED ON CUSTOMER PREMISES DATA

(71) Applicants: Eaton Intelligent Power Ltd., Dublin 4 (IE); Vinya Intelligence Inc., Cleveland, OH (US)

(72) Inventors: Ramanath Ramakrishnan, Westlake, OH (US); Michael E. DeSa, Rye, NY (US); Michael John Froehlich, Farmington Hills, MI (US); Johnie Rose, II, Cleveland Heights, OH (US); Craig Arnold, Cleveland, OH (US); Sunnybhai Patel, Oak Park, MI (US); Aisha Yousuf, Troy, MI (US)

(21) Appl. No.: 16/581,578

(22) Filed: Sep. 24, 2019

Related U.S. Application Data
(60) Provisional application No. 62/736,097, filed on Sep. 25, 2018.
(60) Provisional application No. 62/736,101, filed on Sep. 25, 2018.

(51) Int. Cl. G16H 40/63; G06N 5/02; G06N 20/00

(52) U.S. Cl. CPC G16H 40/63; G06N 20/00; G06N 5/02

(57) ABSTRACT

A system comprises a server computing device comprising a processor and a memory. The memory stores instructions executable by the processor to receive data from one or more devices in a customer premises indicative of a physical state of an occupant of the customer premises; apply one or more predictive models to the data from the customer premises to correlate output from one or more predictive models to an occupant condition; and actuate an action based on output of applying the one or more predictive models.

### CROSS-REFERENCE TO RELATED APPLICATIONS

This application claims priority to U.S. Provisional Application No. 62/736,097, filed Sep. 25, 2018, and U.S. Provisional Application No. 62/736,101, filed Sep. 25, 2018, which applications are each hereby incorporated herein by reference in their entireties.

### BACKGROUND

It is a problem to proactively address risks and potential problem conditions that may confront occupants of premises such as a home or business. For example, an occupant may suffer from a latent or chronic health condition. However, detection and/or prediction of conditions such as chronic illness deterioration can be challenging. Chronic illness deterioration (CID) is defined as an exacerbation of the manifestations of a chronic disease (e.g. chronic obstructive pulmonary disease, congestive heart failure, chronic liver disease, etc.) such that the affected individual suffers increased symptoms and/or decreased ability to function at their baseline. For example, biometric sensors can be provided for an occupant of a home, but such sensors are of limited efficacy; e.g., they are typically only useful if properly deployed, e.g., if an occupant takes active steps to use and/or enable reporting by such sensors. In another example, a premises may be subject to safety or security risks that might go undetected, e.g., due to a leak in a gas or water pipe, an overheating appliance, etc. Again, sensors can be deployed, but use may be limited by costs and the failure of active user action to install, maintain, and monitor such sensors. Accordingly, it is a problem to use data in a premises to detect and/or predict risks and/or potential problem conditions, and to take action to address such conditions.

### BRIEF DESCRIPTION OF THE DRAWINGS

FIG. 1 is a block diagram of an exemplary data distributed digital data management and action system.

FIG. 2 is a flowchart illustrating an example process to trigger and perform an action based on customer premises device data.

FIG. 3 is a block diagram of an example data flows and algorithms in the system of FIG. 1.

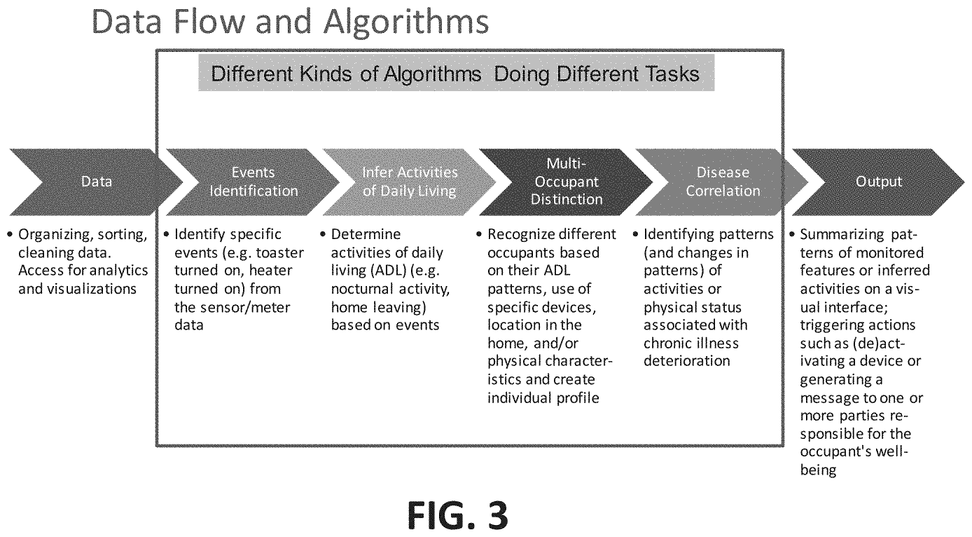

FIG. 4 illustrates an exemplary data analysis in the system of FIG. 1.

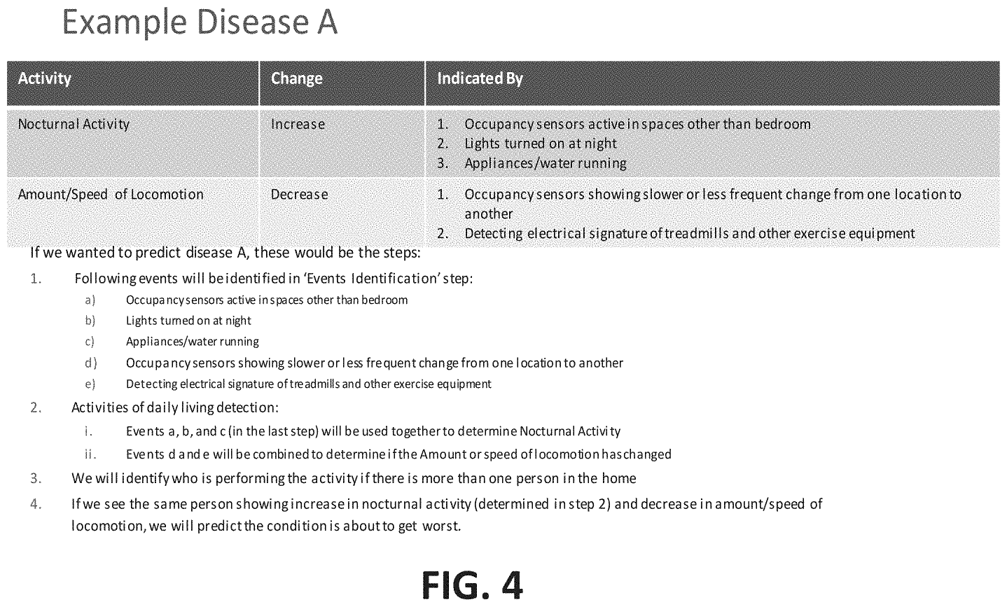

### DESCRIPTION

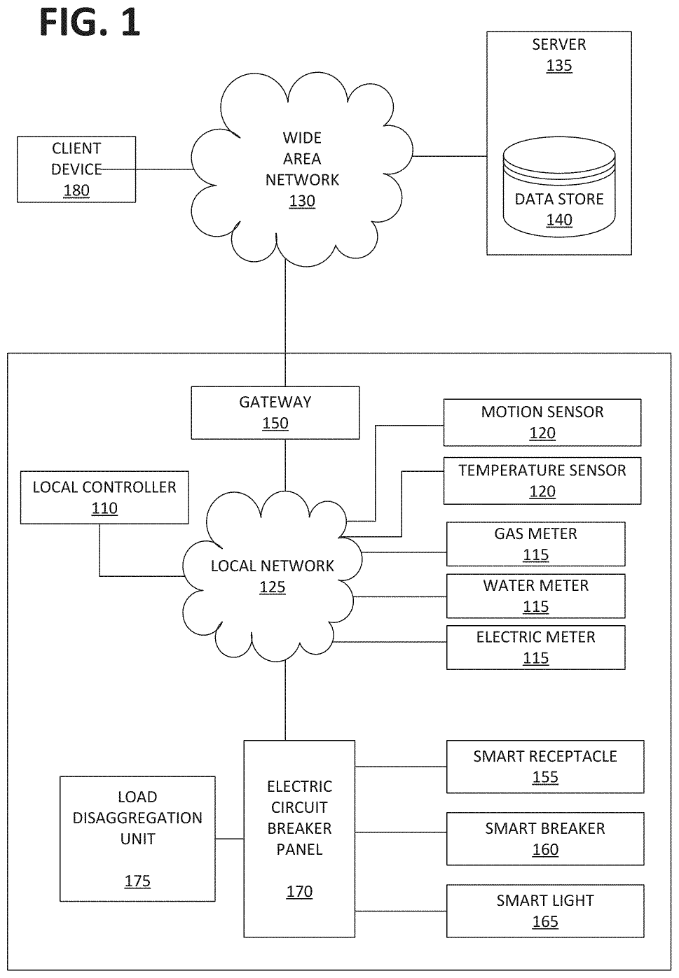

FIG. 1 is a block diagram of an exemplary distributed digital data management and action system 100. The system 100 can obtain, analyze and act on data in a manner that is non-intrusive (i.e., the system 100 may avoid sensors that raise concerns of eavesdropping or monitoring people, such as microphones and cameras), passive (i.e., the system 100 may require no active input from users), and infra-structure based (often using infrastructure elements already present in a customer premises. As seen in FIG. 1, the system 100 includes a customer premises 105. Although the system 100 could, and typically does, include many customer premises 105, only a single customer premises 105 is shown in FIG. 1 for ease of illustration. The customer premises 105 includes a local controller 110 that can communicate with various devices in the customer premises 105 via a local network 125 (i.e., a network internal to the customer premises 105). For example, the local controller 110 can receive and interpret data from, and/or control one or more data collection devices, such as utility meters 115, sensors 120, and/or electrical infrastructure components 155, 160, 165. Data from one or more of the data collection devices 115, 120, 155, 160, 165 can be used classify human activities and to detect or predict changes in health status of a human occupant of the customer premises 105; although detection of health conditions is discussed to illustrate examples herein for ease of exposition, many other examples are possible (e.g., detecting safety and security issues, etc.).

The local controller 110 transmits received data, e.g., from data collection devices 115, 120, 155, 160, 165, to the remote server 135 via a gateway 150 provided for communicating via a wide area network 130 with a remote server 135. The remote server 135 includes programming for analyzing data from the local controller. For example, the remote server can include a combination of supervised and unsupervised machine learning programs. Using machine learning programs or other data analysis techniques or algorithms, the server 130 can process data from a customer premises 105 to identify abnormalities in the behavior or physical status of a human occupant.

For example, the server 135 can be programmed to identify the manifestations from detected abnormalities, patterns, and/or conditions in customer premises 105 data, early manifestations of chronic illness deterioration (CID) in a human. Such manifestations could include changes in temporal sleep/wake patterns, sleep position, sleep location, locomotion (extent, speed, or characteristics), types and amount of daily activities, performance of activities of daily living (ADL's) or instrumental activities of daily living (IADL's), toileting patterns, vital signs (e.g. respiratory rate, heart rate, or body temperature), patterns of home-leaving or socializing in the home, etc. The server 130 can be programmed to recognize changes in behavioral risk factors for disease or disease progression such as smoking, lack of physical activity, or poor diet. The server 130 can be programmed to recognize indirect indicators of disease states such as changes in the use of electrical home health equipment (e.g. nebulizers, oxygen concentrators, constant positive airway pressure (CPAP) machines) or increased detection of airborne particulates consistent with increased concentrations of inhaled medications in the ambient air.

The server 130 can be further programmed to implement one or more actions upon detecting a trigger condition, pattern, etc., in customer premises 105 data, such as activating a further monitoring device, activating a switch or valve, turning a device on or off, initiating an message to a healthcare provider, caretaker, emergency services, etc.

Advantageously, the system 100 can provide early warning, detection, and remediation concerning a premises 105 occupant's well-being. Importantly, the system can do so using predominately passive monitoring, meaning that no direct participation or volitional input on the part of occupants is required, and no equipment need necessarily contact the occupant physically. Remediation of risk conditions means that the system can take action such as described here. Thus, risk conditions can be addressed where they otherwise may not be and/or would not have been addressed in time for improved remediation.

### Exemplary System Elements

The customer premises 105 generally includes a building, structure, or portion thereof, including data collection devices such as the devices 115, 120, 155, 160, 165 discussed herein. For example, the customer premises 105 may be a house, condominium, apartment, a place of business, a public office, or other types of premises 105.

The local controller 110 is a computing device including known hardware and software components, such as a processor, a memory, an operating system, wireless and/or wired network capability, etc. The local controller 110 may be one or more of various computing devices that can include hardware and/or software for operating as described herein. As mentioned above, the local controller 110 communicates with, and receives data from, one or more data collection devices. For example, the local controller 110 may communicate with the data collection devices 115, 120, 155, 160, 165 via the local network 125 using known mechanisms, e.g., according to IEEE 802.11, including WIFI®, ZigBee, Z-WAVE®, BLUETOOTH®, and/or a wired local area network (LAN), e.g., using Ethernet, etc. Further, the local controller 110 is typically programmed to parse and organize data from data collection devices prior to transmission of the data to the server 135. For example, devices 115, 120, 155, 160, 165 typically provide a timestamp associated with each datum, or with a set of data. Devices 115, 120, 155, 160, 165 may further provide metadata indicating a type of data, e.g., a switch status, and electrical load, a measurement of resource (e.g., electricity, water, gas) consumption, etc. The controller 110 can receive data from various data collection devices, e.g., devices 115, 120, 155, 160, 165, and transmit the data to the server 135 with a timestamp or timestamps, and appropriate metadata indicating a particular data collection device and/or type of data being transmitted. Alternatively or additionally, the controller 110 could retain data from devices 115, 120, 155, 160, 165, 170, etc., and could be programmed in addition to or in lieu of programming provided on the server 135 to detect conditions in a customer premises.

Data collection devices, as mentioned above, may include meters 115, sensors 120 and/or electrical infrastructure components 155, 160, 165, each configured to provide data (e.g., having hardware for receiving and transmitting data, and being programmed to provide the data) to the local controller 110. The data collection devices discussed herein include the ability to communicate with the controller 110 and/or each other via the network 125, e.g., via one or more of ZigBee, Bluetooth, WiFi, Ethernet, etc. Additionally, the data collection devices 115, 120, 155, 160, 165 may have further automation and/or control capabilities; in just one possible example, a light fixture or a plug receptacle may be include hardware and/or programming to turn a switch on and/or off based on data received from a motion sensor 120.

Meters 115 can include a gas meter, a water meter, and an electric meter, which may provide information relating to natural gas usage, water consumption, and electricity consumption, respectively, i.e., a utility meter 115 is typically provided to monitor and record a quantity of a commodity (e.g., electricity, water, gas) that is consumed per specified unit(s) of time. Additionally, each meter 115 can report usage or consumption data to the local controller 110 via the local network 125, as well as the remote server 135 via the gateway 150 and external network 130. For example, an electric meter 115 could provide electricity consumption data as kilowatt hours, a water meter could provide consumption data in terms of volume of water consumed in a particular time range, and a gas meter likewise could provide consumption data in terms of volume of gas consumed in a particular time range. Further, in addition to consumption data, more granular or detailed data could alternatively or additionally be provided, such as, for electricity or water, a rate of consumption over time, a frequency of consumption (e.g., a number of discrete times in a time period such as a day when water or electricity in consumed from a faucet or electrical outlet), a rate of ramp-up of consumption, as well as a specific electrical load pattern from an outlet or circuit (e.g., amount of current drawn, current dips or spikes, voltage over time, etc.)

Sensors 120 can include motion and temperature sensors as shown in FIG. 1, and/or can include other sensors such as video and still cameras, noise sensors accelerometers, air quality sensors, smoke detectors, pressure sensors, infrared sensors, etc. Sensors 120 can also include devices which emit electromagnetic waves and sense reflected electromagnetic waves (e.g. radar). Sensors 120 can provide a wide variety of data about a customer premises 105 and/or an occupant thereof. For example, sensors 120 can provide data relating to an occupant's location, posture, and/or movement within the customer premises 105, air quality within the customer premises 105, a detected temperature (whether ambient or the surface temperature of an occupant or portion thereof) within the customer premises 105, whether furniture is occupied and/or experiencing motion (e.g., weight and/or accelerometer sensors), whether a door or window is open or closed, whether a toilet has been flushed, etc. Sensors 120, e.g., a particulate matter sensor, one or more sensors detecting gases produced by combustion, a temperature sensor, a humidity sensor, etc., could provide data about an environmental state or states, e.g., pertaining to air quality, within a premises 105. In general, sensor 120 data is typically associated with a time stamp (or range of time stamps) indicating when a particular datum was, or data were, recorded.

Sensors 120 may further include devices to provide biometric data about a premises 105 occupant. Such sensors could include a weight sensor or digital scale, a blood pressure monitor, a glucose monitor, a heart rate monitor, etc. For example, monitoring an occupant's present weight, changes in weight, blood pressure and changes in blood pressure, and/or changes in other biometric characteristics can be relevant to predicting risk or danger conditions.

Electrical infrastructure components can include, by way of example and not limitation, a smart receptacle 155, a smart breaker 160, a smart light 165, an electrical breaker panel 170, and a load disaggregation unit 175. Table 1 below lists examples of sensors 120 and electrical infrastructure components 155, 160, 165. 170, 175, etc., along with output data from each, that could be used to predict or determine conditions in a premises 105. The sensors 120 could provide other data if needed; Table 1 is for purposes of example and not limitation; these data are just a sample and do not cover all data that could be output from sensors 120.

TABLE 1

| Devices | Output data |
|---|---|
| Energy Management Breaker OR | Voltage (phase 1 & phase 2) |
| Energy Management Receptacle OR | Current (phase 1 & phase 2) |
| Electrical Main Meter | Cumulative energy usage |
| | Time stamp |
| Occupancy Sensors | Event-motion tag |
| | Time stamp |
| Air quality sensor | Carbon (CO & CO₂) |
| | Dust particulates |
| | Humidity |
| | Temperature |
| | Time stamp |
| Water meter | Water flow pulse, time stamp |
| Gas meter | Gas flow pulse, time stamp |
| Respiration sensor | Breathing rate, time stamp |
| Toilet flush sensor | Toilet flush pulse, time stamp |
| Door/window Accelerometer | Motion tag, (x, y, z acceleration), |
| | time stamp |

These components, shown in FIG. 1, are exemplary and not limiting with respect to electrical infrastructure components that could be included in the system 100. All of the components 155, 160, 165, 170, 175 include hardware (e.g., processors and memories) and/or software to provide data via the local network 125 to the controller 110. Examples of specific data that can be provided by each of the components 155, 160, 165, 170, 175 are discussed in turn below, along with other example operations that may be performed or provided by each of the respective components 155, 160, 165, 170, 175.

A smart receptacle 155 may plug into, or be provided as a substitute for, a standard wall outlet (e.g., in the United States, providing electricity according to a standard of 120 Volts and 15 Amperes). A smart receptacle 155 provides the functionality of, but differs from, a traditional or “dumb” receptacle in that it includes a processor and a memory to control provision of power from the smart receptacle 155 and/or to and report the power consumed via the smart receptacle, e.g., via the local network 125. The smart receptacle 155 may wirelessly transmit data, such as power consumption, load status (i.e., whether power is being provided through the receptacle 155 at a particular time or times), etc., to the local controller 110. Further, the local controller 110 can be programmed to not only receive data from, but to control the smart receptacle 155, e.g., to al low a user to turn power on and off at the smart receptacle 155, a client device 180.

A smart breaker 160 includes functionality of a traditional circuit breaker, and in addition can monitor and report on electric power usage, e.g., current load, on an electrical branch circuit connected directly to the breaker 160.

A smart light 165 typically allows a user to control a light fixture, e.g., dim a light, turn a light on or off, etc., via the local controller 110 and/or a client device 180. Further, the smart light 165 can provide data about a status of one or more associated light fixtures, e.g., an on/off status, an amount of brightness being provided, etc.

An electric circuit breaker panel 170 includes the traditional functionality of a circuit breaker panel to distribute an electric power supply into subsidiary electrical circuits within the customer premises 105. The circuit breaker panel 170 can monitor and report on circuit breakers included in the panel 170, including smart breakers 160; the electric circuit breaker panel 170 typically monitors and records the electric power supply to each subsidiary electrical circuit. Additionally, the electric circuit breaker panel 170 protects subsidiary electrical circuits within the customer premises 105 from damage by turning off the electric power supply to an affected subsidiary electrical circuit when an overcurrent and/or an overload occurs. The electric circuit breaker panel also monitors and records when an overcurrent and/or overload occurs, leading to a breaker being triggered.

A load disaggregation unit 175 identifies a specific device or devices, e.g., home appliances, that are active in a customer premises 105, and can identify and report how much power (e.g., voltage and/or current) each identified device is consuming at a specific time. As is conventionally understood, a load disaggregation unit 175 operates by identifying different types of loads, e.g., resistive loads, reactive loads, etc., to associate loads in a customer premises with specific devices, e.g., home appliances.

A gateway 150 generally includes software and/or hardware such as is known for allowing one or more computing devices, e.g., the local controller 110, to communicate via the external network 130. In general, when communications to and from the local controller 110 are described herein, it is to be understood that such communications generally occur via the gateway 150, which is communicatively coupled to a local network 125 and the wide area network 130.

The external or wide area network 130 is one or more mechanisms outside of the customer premises 105 for providing data to and from the gateway 150. Accordingly, the wide area network 130 may be one or more of various wired or wireless communication mechanisms, including any desired combination of wired (e.g., cable and fiber networks) and/or wireless (e.g., cellular network, satellite network, etc.) communication mechanisms, and any desired network topology (or topologies when multiple communication mechanisms are utilized). Exemplary communication networks include wireless communication networks, local area networks (LAN) and/or wide area networks (WAN), including the Internet, etc. The network 130 generally utilizes digital and/or packet networking technologies.

As mentioned above, one or more client devices 180 can access the server 135 to obtain data from the local controller 110. Alternatively or additionally (although not shown in FIG. 1), a client device 180 can access the local controller via the wide area network 130 and/or the local network 125. A client device 180 can be any computing device that can access a digital network 125, 130, and that includes a processor and a memory for performing operations ascribed herein to the client device 180. For example, a client device 180 can include a laptop or desktop computer, a tablet computer, a smart phone or personal digital assistant, etc.

The remote server 135 may be one or more computing devices including a processor, a memory, and a data store 140 and configured for operations as disclosed herein. For example, the remote server 135 may be configured to receive data from the local controller 110 via the wide area network 130.

The remote server 135 typically includes or is communicatively coupled to (using known digital couplings and/or networks) a data store 140. Data provided from one or more local controllers 110 in one or more customer premises 105 can be stored in a data store 140. The data store 140 may include one or more databases or the like such as are known, e.g., relational databases or the like. The data store 140, as explained above, generally includes information associating a customer premises 105, e.g., via a local controller 110 identifier or the like, with set of data. Further, the data store 140 generally stores a location, e.g., a street address and/or geo-coordinates, of a customer premises 105 in association with the unique or substantially unique identifier for the customer premises 105.

The remote server 135 includes machine learning programs, and/or other predictive models or algorithms could be used, e.g., rule-based reasoning, case-based reasoning, etc. For example, a machine learning program can be used to discover and/or map various elements in a customer premises 105, e.g., specific devices or appliances connected to a specific receptacle 155. A machine learning program can further include a chronic illness deterioration (CID) recognition/prediction program. As is known, machine learning programs use a variety of techniques, such as neural networks, to train algorithms to arrive at a predictive model, e.g., a function associating input data with a result or prediction. For example, customer premises 105 data indicating patterns of behavior and/or physical status change corresponding to known CID conditions can be used to train a model or models to predict specific CID conditions based on device 115, 120, 155, 160, 165, etc., data, e.g., examining an electrical load on a receptacle 155 can determine whether usage of an appliance in the customer premises 105, or lack thereof, indicates a specific condition, e.g., a deviation in typical usage of a receptacle 155 mapped to a hair dryer, e.g., an atypical load on the receptacle 155 at a specified time of day, possibly in combination with other data, e.g., from a second receptacle 155 mapped to a microwave oven or coffee maker at a time of day, can predict a CID condition that may warrant action. Then, the server 135 can receive customer premises 105 data from a controller 110, and can retrieve from the data store 140 a model for the specific customer premises 105. The received data can be applied to the retrieved model to determine whether a CID condition is indicated.

Specifically, recognition of a CID condition includes two major steps: 1) classification of human activities based on recognition of associated patterns of sensor signals (e.g. labeling a combination of sensor outputs as representing either sleeping, walking, sitting, eating, coughing, toileting, bathing, hygiene, relaxing, cleaning, etc.), and 2) inputting labeled activities, along with other sensor-derived inputs representing occupant physical status (e.g. surface temperature, respiratory rate, heart rate, sleep position, etc.) into a machine learning algorithm which recognizes aberrance in time series data. Either type of algorithm can be trained based on data from a population of occupants in different premises, but can be differentially optimized (“tuned”) for an individual based on the inputs associated with the individual.

Further, the server 135 can be programmed, upon identifying a particular CID condition, to take one or more specified actions, e.g., activating or deactivating a device or devices in the customer premises 105 (e.g., HVAC, lights, etc.), transmitting a message to a provider of assistance such as a medical provider, transmitting a message to law enforcement or rescue services, etc.

The server 135 can further include programming to provide a user interface, e.g., a “dashboard,” that provides a user of a client device 180 ability to monitor data from a customer premises 105 controller 110. For example, data could be aggregated according to various devices 115, 120, 155, 160, 165 in a customer premises 105 and/or according to types of data, times of day, days of week, etc. For example, power consumption of a particular device or set of devices on an hourly basis for a given day could be graphed. In another example, a graph could indicate an amount of water consumed over time, or at particular times, by toilet flushes. In still another example, time series graphs could indicate sleep/wake patterns, vital signs, or other indicators of activity or physical status over a given time interval. Many other examples are possible. EXEMPLARY PROCESS

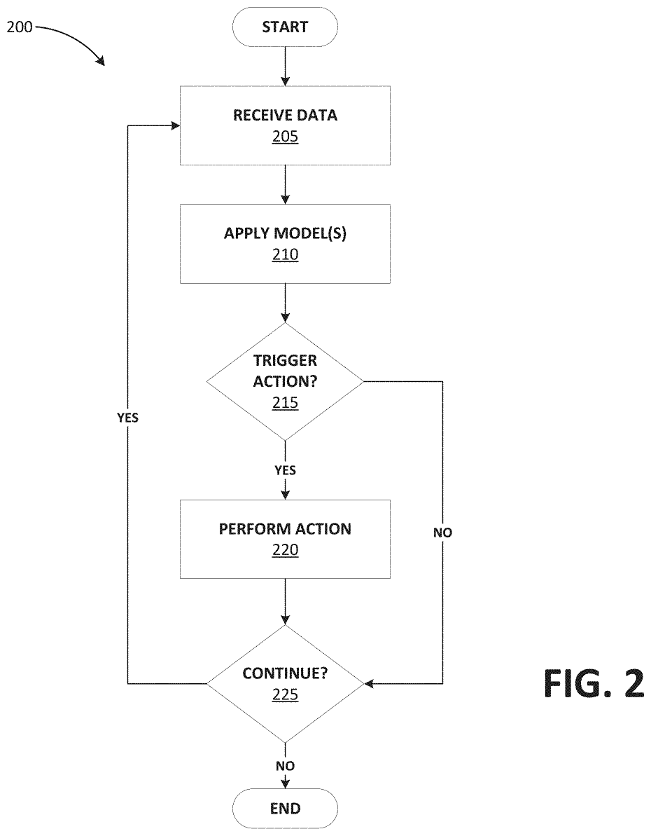

FIG. 2 illustrates an example process 200 to trigger and perform an action based on device 115, 120, 155, 160, 165, etc., data provided from a controller 110 to the server 135. Accordingly, steps of the process 200 may be performed according to programming in the server 135, although as mentioned above alternatively or additionally steps could be performed in the controller 110 or some other computing device in a customer premises 105.

The process 200 begins in a process block 205, in which the server 135 receives data from a controller 110 in a customer premises 105 via the wide area network 130. The data can be time-stamped device 115, 120, 155, 160, 165, etc., data as described above.

Next, in a process block 210, the server 135 applies one or more predictive models (e.g., machine learning models) or functions to the data received in the block 205. The server 135 may be programmed to execute the block 210 on a periodic (e.g., multiple times per hour) or substantially continuous, or some other basis (e.g., at a time when a specified amount of data has been accumulated), and/or could be included in a set of parallel processes executing different predictive models, to analyze, i.e., apply the predictive model or function to, the received data from a particular customer premises 105. As explained above, the predictive model or function may be obtained via a variety of supervised and unsupervised machine learning techniques, such as a neural network, Bayesian analysis, a decision tree, etc.

Next, in a decision block 215, the server 135 analyzes output from application of the one or more predictive models or functions to determine if an action is triggered. That is, if output of a model indicates a risk or danger condition, e.g., a CID condition, then the server 135 determines that an action is triggered, and proceeds to the block 220. The server 135 may obtain from the data store 140 a table or the like indicating outputs or ranges of outputs from applying a predictive model or function that are respectively associated with actions. For example, actions could include transmitting a message to an assistance provider, activating or deactivating a device in the applicable customer premises 105, etc. Otherwise, the process 200 proceeds to the block 225.

In the process block 220, the server 135 performs an action, e.g., transmitting a message or activating or deactivating a device in the applicable customer premises 105. Messages in this context may include any combination of the following: a notification that recent patient activities or physical status have deviated from normal patterns based on output from a machine learning algorithm recognizing aberrance in time series data, a brief summary of data that summarizes the nature of the message and the event or trend triggering it, an indicator of the degree of certainty that the event or trend believed to have occurred has indeed occurred, an indication of the threat to health inherent in the trend or event, probability of unscheduled healthcare utilization (e.g. clinical office visits, emergency department visits, or hospital admissions for worsening symptoms of chronic disease) within a discrete time period (e.g. 50% probability of unscheduled utilization within the next 48 hours), instructions or a hyperlink for obtaining more detailed information about the event or trend triggering the message, and/or a list of other stakeholders who have received an message triggered by the same event or trend.

Messages can be passive or active depending on the potential severity of the events triggering the message. Passive messages are notifications encountered by a user when they voluntarily check an information source (e.g. check their email, log into an electronic health record, log into an internet portal, etc.). Active messages are notifications brought to the attention of users immediately through a mechanism designed to capture the user's attention (e.g. a ‘push’ notification via an application on a mobile device, an SMS text message, a pop-up warning from an electronic health record system). The active message may come in forms including, but not limited to SMS text message, alphanumeric page, email, electronic health record (EHR) notification, mobile push notification, and/or smart speaker/digital assistant prompt. An active or passive message may be provided to one or more users including, but not limited to, the following: the patient themselves, patient's family members (whether co-habitating or otherwise), informal caregivers, home clinical or custodial care providers, chronic disease management personnel, outpatient providers, inpatient providers, individuals responsible for monitoring the health outcomes of populations, accountable care organizations, health systems, and/or health insurers. Another example of a triggered action includes activation of a smart speaker/digital assistant device program which interacts verbally with the occupant to provide one or more reminders (e.g. to take a prescribed medication) or to elicit information from the occupant (e.g. confirmation of symptoms).

In a decision block 225, which may follow either the decision block 215 or the process block 220, the server 135 determines whether to continue the process 200. The decision block 225 could be located at other points of the process 200, e.g., a user could provide input at any time to terminate the process 200, the process 200 could be programmed to terminate at a specified time, the server 135 could be powered off or lose power, etc. In any event, if the process 200 is to continue, control returns to the block 205. Otherwise, the process 200 ends.

The disclosure has been described in an illustrative manner, and it is to be understood that the terminology which has been used is intended to be in the nature of words of description rather than of limitation. Many modifications and variations of the present disclosure are possible in light of the above teachings, and the disclosure may be practiced otherwise than as specifically described.

What is claimed is:

1. A system, comprising a server computing device comprising a processor and a memory, the memory storing instructions executable by the processor to:
receive data from one or more devices in a customer premises indicative of a physical state of an occupant of the customer premises;
apply one or more predictive models to the data from the customer premises to correlate output from one or more inference or predictive models to an occupant condition; and
actuate an action based on output of applying the one or more predictive models.

2. The system of claim 1, wherein the one or more devices include at least one of a gas meter, a water meter, and an electricity meter, and the data includes a measurement of at least one of gas, water, or electricity.

3. The system of claim 1, wherein the one or more devices include a motion sensor, and the data include data indicating motion from the motion sensor.

4. The system of claim 1, wherein the one or more devices include a switch, and the data include data indicating activation of the switch.

5. The system of claim 1, wherein the one or more devices includes a smart receptacle that includes an electrical receptable, a processor, and a memory, the memory storing instructions such that the processor is programmed to control power consumption via the receptable, and to store data specifying the power consumption via the receptable, wherein the data specifying power consumption via the receptacle is included in the customer premises data provided to the server.

6. The system of claim 1, wherein the one or more devices include a load disaggregation unit and a plurality of appliances, wherein the customer premises data includes respective electrical loads for at least some of the plurality of appliances.

7. The system of claim 1, wherein the data is provided from at least one of the one or more devices without user input.

8. The system of claim 1, further comprising a computing device in the customer premises and comprising a processor and a memory, the memory storing instructions executable by the processor to receive aggregate data from the one or more devices and provide the aggregated data to the server; wherein the server applies the one or more predictive models to the aggregated data.

9. The system of claim 1, wherein the one or more inference or predictive models is at least one of rule-based, case-based, or developed by machine learning.

10. The system of claim 1, wherein the one or more predictive models is a machine learning program that includes a chronic illness deterioration (CID) prediction program.

11. The system of claim 10, wherein the one or more predictive models correlates a specific CID condition to the data from the one or more devices.

12. The system of claim 11, wherein the specific CID condition is specific to a specific occupant of the customer premises.

13. The system of claim 1, wherein the action includes activating or deactivating a device or devices in the customer premises.

14. The system of claim 1, wherein the action includes transmitting a message.

15. The system of claim 14, wherein the message includes at least one of a notification that occupant status has deviated from a normal pattern, a description of an event or trend triggering the message, or an indication of a threat to health.

16. The system of claim 1, wherein the occupant condition is specific to a specific occupant of the customer premises.
